(19) United States
(12) Patent Application Publication
(10) Pub. No.: US 2002/0192123 A1
(43) Pub. Date: Dec. 19, 2002

(54) Heat-regulating container for atmosphere conditioning systems

(76) Inventor: Yong S. Chen, Pleasanton, CA (US)

Correspondence Address: Ray K. Shahani, Esq., Attorney at Law, Twin Oaks Office Plaza, 477 Ninth Avenue, Suite 112, San Mateo, CA, 94402-1854, US

(21) Appl. No.: 09/870,115

(22) Filed: May 30, 2001

(51) Int. Cl. A62B 7/08; B01D 5/00; A61L 9/00

(52) U.S. Cl. 422/125; 422/244; 422/305; 422/306; 422/307

(57) ABSTRACT

A heat-regulating container for use with a heating system, the container having integral leg support structure to provide temperature step-down between the heating apparatus and the container, the leg supports in the form of a plurality of individual dimples or bumps, in order to achieve desirable temperature for the controlled release of active ingredients such as pesticides, disinfectants and fragrances contained in the reservoir of the container, the system being used for the efficient conditioning of an atmosphere, such as an office, workplace, industrial or household room.

### FIELD OF THE INVENTION

A temperature or heat regulating container having a built-in structure to control temperature within the container, the container used as a reservoir in conjunction with heating apparatus to achieve controlled release of volatile active ingredients, such as pesticides, disinfectants and fragrances, for any type of atmosphere conditioning systems.

### BACKGROUND OF THE INVENTION

Mosquitoes and other insects can be more than a simple nuisance, as they are vectors of deadly diseases like Dengue, Haemorrhagic fever, Malaria, Yellow fever, and various types of Encephalitis. Protecting one's self and family from these diseases is, thus, a premium concern. Devices that repel and kill mosquitoes abound in the market place. There are three basic types of anti-mosquito devices: aerosol insecticides, coils, and electrical devices like mats and emanators.

Aerosols dispense oil or water based, insecticide containing droplets into the air which impact on the mosquito, delivering the insecticide and killing the insect. These droplets impact on the mosquitoes and either repel them or kill them depending upon how many droplets the insect such as a mosquito encounters. Aerosols are effective for a period of time from between about a few minutes to about a few hours.

Coils are typically composed of pressed sawdust impregnated with a small amount of insecticide. The coil is lit with a match and begins to smolder slowly. As it smolders, the heat evaporates the insecticide into the air as a vapor where it quickly cools and forms micron sized droplets.

Electrical devices like mats and emanators use electrical heat instead of smoldering sawdust. Mats are often composed of an insecticide and a carrier or solvent impregnated into cotton linter that are placed on small metallic heating plates or trays which drive off the active ingredient into the air in the same manner as the coils. Mats can last from between about 4 to about 15 hours.

Emanators work much the same way, but they use a liquid reservoir which contains insecticide and a carrier solvent. The insecticide is carried up to the heating element by a wick, usually ceramic. Because the reservoirs are large, emanators can last up to 90 days.

Electrically powered flying insect killers, like other emanator devices have been growing in popularity for some time. Emanators are preferred over aerosols because of, inter alia, the convenience of longer operating times without re-spraying or re-charging, and their odorless operation. They are safer than coils, with their drastically reduced fire risk, and cleaner because there are no messy ashes or smoke to deal with. They are typically perceived of as more modem, upscale products, preferred over seemingly low-tech coils and aerosols.

An improvement over the above mentioned devices is described in co-pending U.S. patent application Ser. No. 470,136, issued to Schiavo et al, which describes a method for the metered delivery of an insecticidal liquid where the liquid is ejected in small quantities from a bubble-jet type device, without utilizing the traditional methods of heat. By passing the ejected droplets through a static field they can be imparted with a charge attractive to the insects cuticle, attracting them to the insect at room or ambient temperature.

It is known in the prior art to impregnate a solid porous mat with a volatile material, or to place a volatile material in a pan-like metal structure. These mats and pans are placed on heaters to cause the volatile material to vaporize into the atmosphere. U.S. Pat. No. 4,439,415 is a general discussion of heater units used for this purpose. This patent is incorporated herein by reference in its entirety, as if fully set forth herein.

A problem with such metal pans is that for typical heaters they cause the volatile material to be exposed to too much heat. This causes the volatile material to be used up too fast or to degenerate or decompose prematurely, particularly during extended usage.

Mats have similar and significant problems with respect to being exposed to different temperatures across the mat surface. In this regard, low-cost, existing heaters often have hotter regions at certain points along their burner surface, resulting in hot spots and cool regions. The mats therefore have somewhat inefficient vaporization.

These problems are of increased concern for products designed for use for a week or more. Merely adding additional volatile material does not adequately address the problem as prolonged exposure of volatile materials to high temperatures wastes or is otherwise an inefficient use of the volatile materials.

Another design consideration is that existing heaters, for safety and other reasons, often only accept mats or other inserts having a small cross-sectional shape so that they can fit into a small heater opening. Utilizing a low profile opening is safer and avoids safety risks when operating in households or other areas where children may be prone to play with such devices Thus, the parameters of any solution to the above problems need to take into account these overall size restrictions for such containers.

FIG. 1 shows a lover view of a container 100 of the prior art. FIG. 1 shows a type of container 100 of the prior art with a reservoir for volatile materials used for this purpose. It is sold by S. C. Johnson & Son, Inc. under the trademark FUTYI VAPE. This product is described in International Application No. WO 00/67574, by Flashinski et al. The device 100 has a reservoir 106 which contains the volatile material. An insulating pad 104 surrounds the reservoir 106 for controlling the temperature and rate of dispensing of volatile materials contained in the reservoir 106. The container 100 is held at handle portions 102 and inserted into a suitable opening in a small heating device The insulating pad 104 insulates the volatile material in the reservoir 106 from direct exposure to heat. However, the insulating pad 104 is made of a different material than the reservoir 106 itself, it must be applied to the reservoir 106 in additional manufacturing steps, and can come loose inadvertently or be removed unintentionally which would either interfere with or eliminate completely the benefit of the insulating pad 104.

As such, it can be seen that a need exists for an improved volatile material dispensing container for use in conventional or specialized heater devices.

### SUMMARY AND ADVANTAGES

A temperature regulating system with a container having structured legs to incorporate a built-in air insulation between a heating apparatus and the container as one unit in order to achieve desirable temperature for the controlled release of active ingredients such as pesticides, disinfectants and fragrances contained in the reservoir of the container, the system being used for the efficient conditioning of the atmosphere in a room.

One object and advantage of the present invention is to provide an integral metal or other heat-resistant material container which incorporates an insulating air gap.

Another object and advantage of the present invention is to provide the air gap created as part of the container structure. Designs which integrate the air gap have a minimum of one (1) or two (2) legs, or more, such as at four (4) corners of a tray or pan-type reservoir, or in a zig-zag design across the lower portion of the reservoir, or other pattern design.

A further object and advantage of the present invention is to provide a system which allows for control within any desirable range of the rate of release of volatile active materials. In a preferred embodiment this can be achieved by varying the length of the legs of the container, i.e., by varying the size or volume of the air gap.

One object and advantage of the present invention is to provide a container made of one or more types of thermally conductive materials such as metal, etc. In a preferred embodiment of the present invention, an aluminum container is used which provides an efficient and economic means to regulate temperature.

A further object and advantage of the present invention is to provide a support between the reservoir of the volatile material and the hot surface of the heater unit and results in a temperature step-down between the hot exposed surface and the contacting surface of the reservoir.

A further object and advantage of the present invention is to provide an air gap integrated into the design of the metal structure which will serve to spread heat more uniformly across the lower surface of the reservoir of the container. This leads to more efficient use of expensive volatiles as well as reduces degradation due to exposure to excessive temperature over prolonged periods. As a result, inserts can be designed for use for a week, a month, or several months.

One object and advantage of the present invention is that the volatile material is selected from the group consisting of insecticides, insect repellents, fragrances, and deodorizers. The volatile or otherwise active material which is to be dispersed into the atmosphere can be in the form of a liquid or gel composition. A solid substrate (such as cellulose) may be positioned in the reservoir, with the substrate being impregnated with the volatile material.

A further object and advantage of the present invention is the cost-effectiveness of the design. Mats of this type are often used in countries that have very modest average annual incomes. To have much practical application in those countries, the mats must be inexpensive.

Numerous other advantages and features of the present invention will become readily apparent from the following detailed description of the invention and the embodiments thereof, from the claims and from the accompanying drawings. The claims should be looked to in order to understand the full scope of the invention.

### BRIEF DESCRIPTION OF THE DRAWINGS

The invention is illustrated in greater detail below with the aid of an example which is represented schematically in:

FIG. 1 shows a lover view of a container 100 of the prior art.

FIG. 2A is a representative view of the heater device environment and apparatus 200 in which the heat-regulating container of the present invention is used.

FIG. 2B is a representative view of an alternate embodiment of the heater device environment and apparatus 200 in which the heat-regulating container of the present invention is used.

FIG. 3 is an upper, perspective view of a preferred embodiment of the heat-regulating container 300 of the present invention.

FIG. 4 is a lower perspective view of a preferred embodiment of the heat-regulating container 300 of the present invention.

FIG. 4A is a lower perspective view of an alternate preferred embodiment of the heat-regulating container 400 of the present invention.

FIG. 5(a) is a section view of a preferred embodiment of the integral support structure 502 of the heat-regulating container 300 of the present invention.

FIG. 5(b) is a section view of another embodiment of the integral support structure 502 in a zig-zag pattern of the heat-regulating container 300 of the present invention.

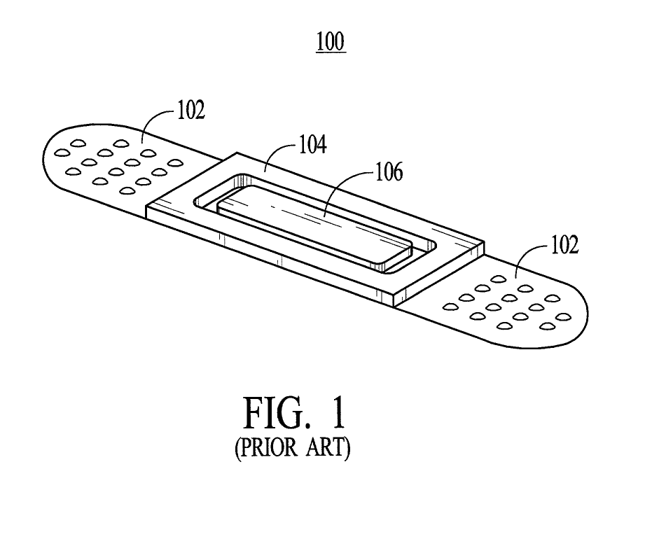

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

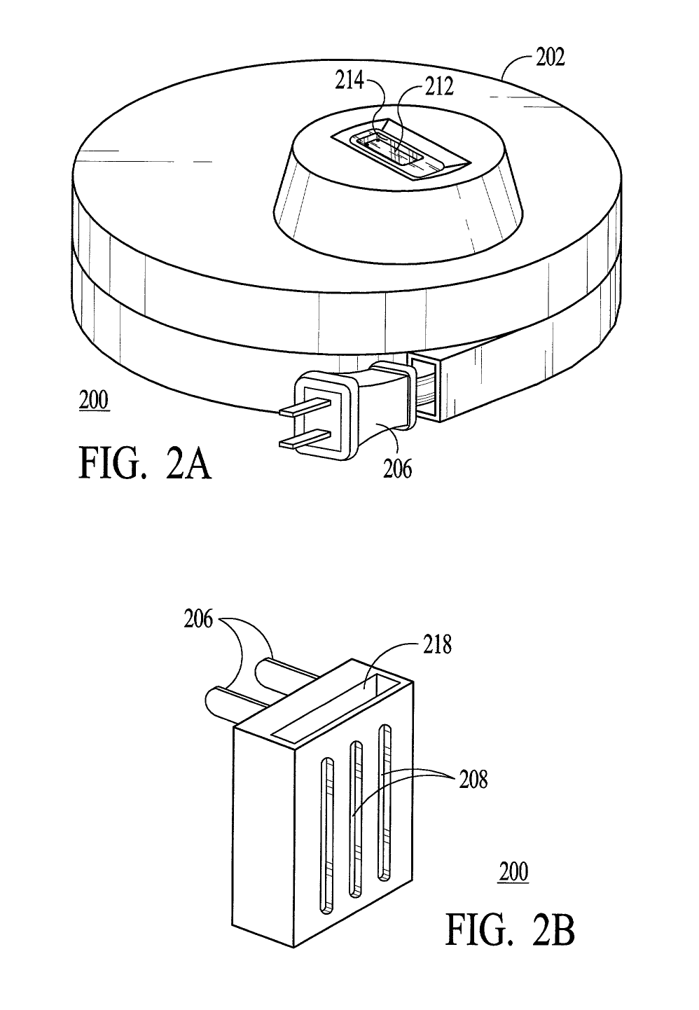

FIG. 2A is a representative view of the heater device environment and apparatus 200 in which the heat-regulating container of the present invention is used. FIG. 2B is a representative view of an alternate embodiment of the heater device environment and apparatus 200 in which the heat-regulating container of the present invention is used. The typical device 200, as shown in FIG. 2A, has a side opening 218 allowing for insertion therein of a heat-regulating container (not shown) of the present invention. The heating apparatus 200 can be any appropriate or suitable electric or other type of emanator or heater, as described above. In a preferred embodiment, the electric heater device 200 is an electrical-resistance heater, and has a flat, upwardly exposed hot surface or heating plate 212 adjacent to which is placed the improved container device 300 of present invention. An electrical connector 206 supplies power to an electrical resistor element (not shown) which creates the hot surface 214. It will be understood that the heater device 200 can also be battery-operated. As shown, safety panels 208 are provided. These safety grid or panel structure 208 can be fixed or, optionally, adjustably oriented to direct the emanating volatile material 308 to any desired direction.

As described herein, the heat-regulating container device 300 of the present invention is adapted for use with a wide variety of electrical and non-electrical heaters 200 available for heating conventional and specialized mosquito mats. It will be understood, however, that the heater device 200 can be replaced by any conventional or specialized, positive temperature co-efficient, or other sources of heat, such as contained flame or catalytic combustion, or other heating means which may be known in the art.

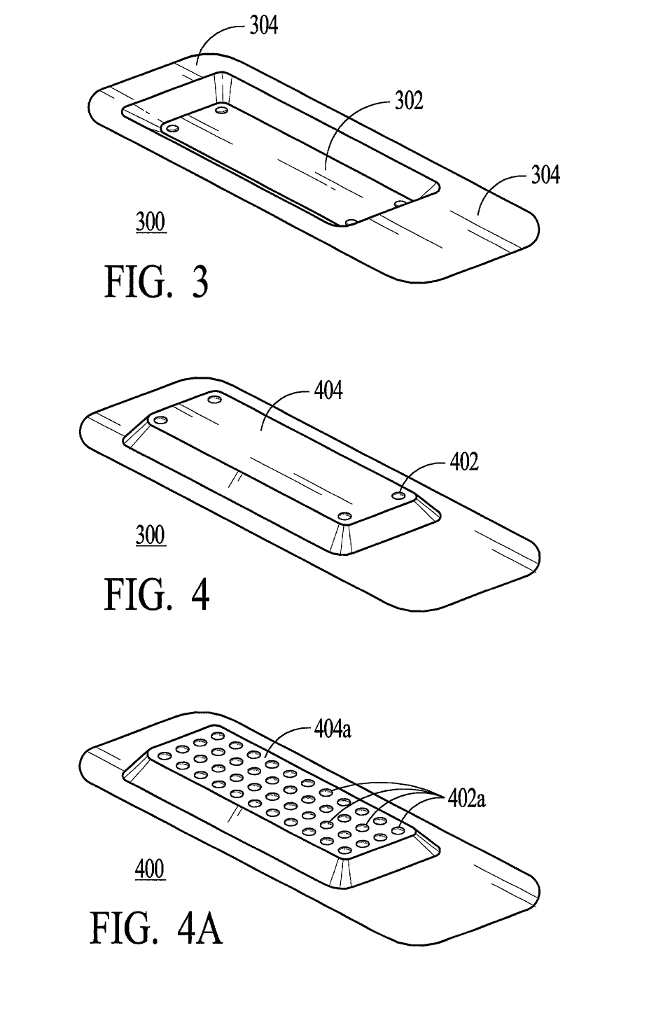

FIG. 3 is an upper, perspective view of a preferred embodiment of the heat-regulating container 300 of the present invention. The container 300 comprises reservoir portion 302 in the central portion of the container 300. The reservoir 302 contains the volatile material 308. The container 300 has gripping tabs or other handle means 304 on either end of the container 300.

The volatile material 308 can be any one or combination of insecticides and insect repellents, and/or other active agents. Particularly preferred are organic phosphorous insecticides, lipidamide insecticides, natural repellents as citronella oil, natural pyrethrins ad pyrethrum extract, and synthetic pyrethroids. Suitable synthetic pyrethroids are allerbrin as Pynamin, d-allethrin as Pynamin forte, benfluthrin, bifenthrin, bioallethrin, S-bioallethrin, esbiothin, esbiol, bioresmethrin, cycloprothrin, cyfluthrin, beta-cypermethrin, cyphenothrin, deltamethrin, empenthrin, esfenvalerate, fenpropathrin, fenvalerate, flucythrinate, tau-fluvalinate, kadethrin, permethrin, phenothrin, prallethrin as ETOC, resmethrin, tefluthrin, tetramethrin, transfluthrin, or tralomethrin.

Deodorizers, such as a terpene based deodorizer fragrance may also be used in the reservoir portion 302 of the container 300 of the present invention. Further, volatile fragrances, disinfectants, or other air quality modifying agents may be used, such as glycols, trimethylene, and dipropylene. In addition, organic acids that are compatible with the use of the substrate and the atmosphere can also be utilized.

It will be understood that a flexible, transparent or semi-transparent, colored or otherwise opaque, permeable or semi-permeable or other type protective and retaining film or membrane 306 may be used to cover the reservoir 302. The membrane 306 or other volatile material retaining structure or other closure means may be completely impermeable, or may have selective permeability, it may allow vapors to pass selectively, it may be temperature responsive or otherwise. The membrane 306 may retain a fluid, gel, mat, solid, saturated substrate or other form of volatile active material to be dispensed into the atmosphere.

FIG. 4 is a lower perspective view of a preferred embodiment of the heat-regulating container 300 of the present invention. Support legs or other means 402 emanate from and integral with lower surface 404 of the container device 300. Lower surface 404 is exposed to heat primarily indirectly but also directly through contact between the support leg means 402 and the hot surface of the heating device, shown best in FIG. 2. In a preferred embodiment, the lower surface 404 is comprised of metal or other material with high thermal conductivity, similar to or different than the material of construction of the rest of the container device 300 of the present invention. The height of the support leg means 402 can be varied, thus providing varying degrees of temperature regulation, step-down or insulation, optionally optimized for the type of insecticide or other volatile material 308 being used, the temperature of the hot surface 204 heating device 200, the rate of delivery desired, etc. The number and pattern of leg support means may vary to achieve the desired results. Any pattern or height of such leg support means 402 can be integrated into the lower surface 404 of the container device 300 of the present invention. An air gap is thus created by the leg support means 402 between the lower surface 404 of the container device 300 and the heating plate 212. This built-in air space serves as a temperature control means. The leg supports 402 the leg supports can also be in the form of a plurality of individual dimples or bumps, or other suitable heat-regulating structure.

The heat and the amount of volatile material 308 must be sufficient to volatilize insecticidal agent material in amounts sufficient to achieve the desired insect control. The specific rate of delivery of volatile material 308 and amount of heat necessary will depend on the particular chemical or composition of insecticidal material used and volume of air space created by the leg support means 402, the rest of the lower surface 404 of the container device 300, and a heating surface 214.

In preferred embodiments of the present invention, designs integrate an air gap having a minimum of one (1) or two (2) leg support means 402, or more, such as at four (4) corners of a tray or pan-type reservoir, or in a zig-zag or other pattern design across the lower surface 404 of the container 300.

FIG. 4A is a lower perspective view of an alternate preferred embodiment of the heat-regulating container 400 of the present invention. In this embodiment of the present invention, a 2-dimensional series or pattern of leg support means 402a across the lower surface 404a of the container 400 provides the enhanced utility and function of the present invention. It will be understood that the leg support means 402a can be stamped or punched into the lower surface 404a of the metallic or other suitable material containers 400.

Additionally, it will be understood that any desired pattern of leg support means 402a can be used, and that while the lower surface portion 404a shown in FIG. 4A shows the entire surface 404a covered with such leg supports 402a, the leg supports 402a could similarly be positioned in discrete strips, swathes, or other patterns over the lower surface.

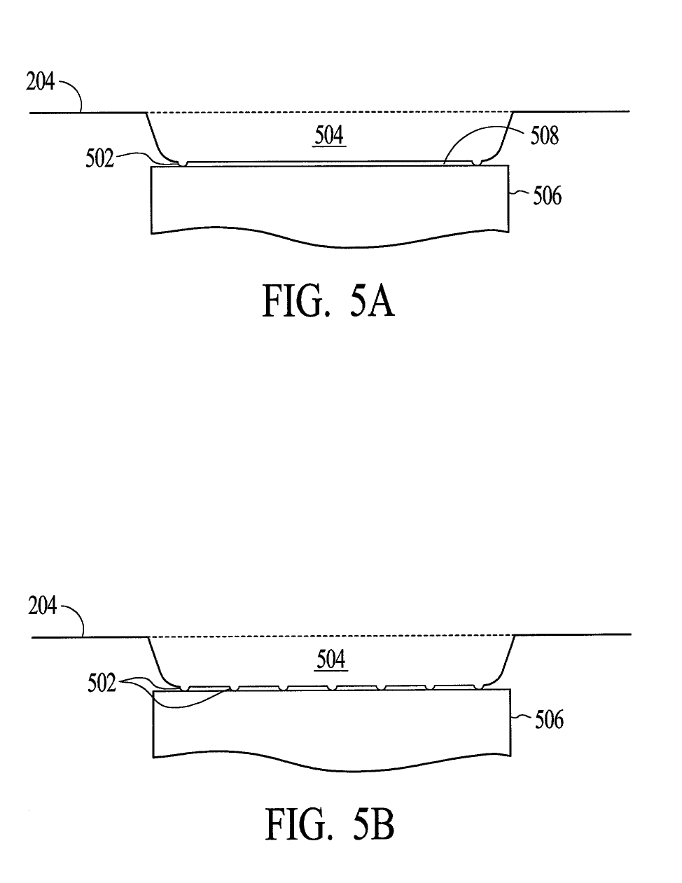

FIG. 5(a) is a section view of a preferred embodiment of the integral leg support structure 502 of the heat-regulating container 300 of the present invention. FIG. 5(b) is a section view of another embodiment of the integral support structure 502 in a zig-zag pattern of the heat-regulating container 300 of the present invention.

FIG. 5(a) shows a container device 300 which has two or more leg support means 502 supporting the container device 300 with reservoir 302 for containing volatile material 308. The height of the leg support means 502 regulates heat transfer between the lower surface 404 of container device 300 by defining the size or volume of air space 508. FIG. 5(b) shows an alternative embodiment of the container device 300 such as shown in FIG. 3 with a zig-zag pattern of the leg support means 502.

It will be understood that the integral leg support means 402 and 502, as shown in FIGS. 4, 5(a) and 5(b) can be integrally molded into the lower surface of the container device, if the container device 300 is constructed of a plate or sheet metal the support leg extrusions can be stamped, pressed, rolled, crimped, or otherwise formed directly onto the lower surface 404 of the container device 300. Leg support structure 402 or 502 or other similar means with essentially any level of thermal conductivity can also be manufactured or formed independently and then adhered or otherwise coupled directly to the lower surface 404 of the container. In this case, the bond or adhesion layer becomes the integral junction between the leg support means 402 and 502 and the lower surface 404 of the container device 300 of the present invention.

Experimental Data

The following Table 1 provides experimentally obtained data directly correlating support leg means 402 height with temperature as measured at the lower surface 404 of the container device 300 of the present invention.

TABLE 1
Experimental Support Height/Surface Temperature Correlation Data

| | Surface |
|---|---|
| Leg Height | Temperature |
| (mils) | (F.) |
| 0 | 165 |
| 4 | 140 |
| 6 | 135 |
| 8 | 130 |
| 10 | 127 |
| 12 | 125 |
| 14 | 121 |
| 16 | 118 |
| 18 | 115 |
| 20 | 113 |
| 24 | 109 |

It will be understood by the foregoing supporting description and data that the operating parameters of the container device 300 of the present invention for controlled delivery of volatile or other evaporative agents into the atmosphere can be selected and integrated into the design of the device 300 precisely and independently, as desired.

Unless defined otherwise, all technical and scientific terms used herein have the same meaning as commonly understood by one of ordinary skill in the art to which this invention belongs. Although any methods and materials similar or equivalent to those described can be used in the practice or testing of the present invention, the preferred methods and materials are now described. All publications and patent documents referenced in this application are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

We claim:

1. A system for controlled dispensation of active ingredients into an atmosphere, the system comprising:
a heat-regulating container made entirely of metal and having one or more reservoir portions, a volatile material comprising active ingredients to be dispensed into the atmosphere contained within the one or more reservoir portions, and a lower surface having integral leg support structure; and
a heating device having a heating surface and adapted to receive the heat-regulating container such that the integral leg support structure is in direct contact with the hot surface, thereby regulating the temperature of the volatile material in the one or more reservoir portions within the container.

2. The system of claim 1, wherein the volatile material is one or more selected from the group consisting of pesticides and insecticides, insect repellents, fragrances, air-fresheners and deodorizers.

3. The system of claim 1, wherein the one or more reservoir portions contains a porous solid substrate positioned within the one or more reservoirs and wherein the volatile material is impregnated within the substrate.

4. The system of claim 1, wherein the volatile material is in a gel form.

5. The system of claim 1, wherein the container is made of a single heat-resistant material selected from the group consisting of metal, thermoplastic, and ceramic.

6. The system of claim 1, further comprising a plurality of integral leg support structures.

7. The system of claim 6, wherein the plurality of integral leg support structures are provided in a zig-zag pattern.

8. The system of claim 6, wherein the plurality of integral leg support structures are provided in a solid pattern over an entire lower surface portion of the container.

9. The system of claim 1, in which the container further comprises handles means extending from the container for manipulation of the container by a user.

10. A heat-regulating container for dispensing volatile materials into an atmosphere, the container adapted for use in a heating device having a heating surface at elevated temperature, the container comprising:
a reservoir portion for containing volatile material to be dispensed;
a lower surface; and
a plurality of integrally formed leg structures extending from the lower surface of the container for regulating the transfer of heat from a heating surface of a heating device to volatile material to be dispensed.

11. The container of claim 10, further comprising a predetermined number of integrally formed leg structures.

12. The container of claim 10, in which the plurality of integrally formed leg structures each have a predetermined height.

13. The container of claim 10, further comprising a closure means for retaining the volatile material in the reservoir portion.

14. The container of claim 13, in which the closure means comprises an impermeable film.

15. The container of claim 13, in which the closure means comprises a semi permeable membrane.

16. The container of claim 13, in which the closure means comprises a permeable membrane.

17. The container of claim 10, further comprising a volatile material.
